(19) United States
(12) Patent Application Publication
(10) Pub. No.: US 2003/0036097 A1
(43) Pub. Date: Feb. 20, 2003

(54) Urea containing reagents for treatment of proteins

(76) Inventor: Aftab Alam, St. Louis, MO (US)

Correspondence Address: AFTAB ALAM, 500 CLAYTON MEADOWS, ST. LOUIS, MO, 63011, US

(21) Appl. No.: 10/218,420

(22) Filed: Aug. 14, 2002

Related U.S. Application Data
(60) Provisional application No. 60/313,387, filed on Aug. 17, 2001.

(51) Int. Cl. G01N 33/53; C07K 1/00

(52) U.S. Cl. 435/7.1; 530/422

(57) ABSTRACT

A protein solubilization reagent made in two parts, of which one part is a dry urea-agent and the second part is a diluent for re-hydration of the dry urea-agent. The urea-agent comprises of urea and one or more agents selected from a group consisting of dry thiourea, detergents, sufobetaines, sulfonates, buffer agents, and dyes. The diluent contain pure water and other agents.

### RELATED PATENT APPLICATION

This patent application claims priority from pending provisional patent application Serial No. 60/313,387, filing date Aug. 17, 2001.

### HISTORY OF THE INVENTION

Urea is used for solubilization and denaturation of protein. One of the disadvantages of using urea is carbamylation. Urea in water exists in equilibrium with ammonium cyanate, the level of which increase with increasing temperature and pH. Cyanate reacts with α-amino group of the N-terminal and the E-amino groups of lysines. This reaction leads to artifactual charge heterogeneity of protein sample. Therefore, carbamylation must be avoided. Because of the risk of carbamylation, the urea based reagents must be prepared fresh for each use. Making fresh reagent for each use is very demanding on research time. Therefore, there is need for developing reagent formulations and the method of delivery of the reagents containing urea that will simplify reagent preparation and also prolong the shelf life of ready-to-use urea based reagents. The present invention relates to a method of preparation of urea based reagents, a chemical composition, an article of manufacture of urea based reagents for protein solubilization.

### SUMMARY OF THE INVENTION

This invention relates to a method, an article of manufacture, and a method of preparation of protein solubilization reagents containing urea. The protein solubilization reagent comprises a reagent in two parts of which the first part is a dry urea-agent and the second part is a diluent for mixing with and re-hydration of the dry urea-agent. The dry urea-reagent comprises urea and may also contain other dry chemical agents non-reactive with urea. The dry urea-agent may contain urea and one or more agents selected from a group consisting of dry thiourea, detergents, sufobetaines, sulfonates buffer agents, and dyes.

The second part of the urea-reagent is a diluent for mixing with and re-hydration of the dry urea-agent. The diluent part comprises water and may contain one or more agent selected from a group consisting of dyes, carrier ampholyte, detergents, buffering agents, glycerol, reducing agents, and alkalation agents.

The protein solubilizaiton reagent is provided with an instruction for mixing and re-hydration of the dry urea-agent with the diluent for the preparation of a working protein solubilization reagent of a desired concentration.

For each gram of dry urea-agent, approximately 1-2 ml of diluent is needed to achieve a desired final concentration of urea, thiourea, and other agents in the re-hydrated protein solubilization reagent.

The amount and the concentration of each agent in both parts, the dry urea-agent and the diluent, are such that on mixing a pre-determined amount of the dry urea-agent with a predetermined volume of the diluent the final concentration of each agent in the re-hydrated mixture reaches a desired concentration, molarity, and pH. On re-hydration of the dry urea-agent the final concentration of urea in the re-hydrated mixture is between 6-9M. If thiourea is added in the dry urea-agent then the final concentration of thiourea in the re-hydrated mixture is approximately 2M.

The dry urea-agent is preferably provided as fine ground powder or as tablet form. The dry urea-agent is provided in a container. The container may be a large bottle or jar and is, preferably, filled less than two-third with the dry urea-agent. Preferably, the bottle or jar container is provided with a marble or a similar solid object for mixing the dry urea-agent before taking out an aliquot of the dry urea-agent for re-hydration with the diluent. The mixing of the dry urea-agent is performed by vigorously shaking the container (bottle or jar); the marble in the container assists mixing of the dry urea-agent. When the dry-urea agent is provided in a bottle or jar, aliquots of the dry-urea agent is taken out and mixed with the diluent on as need basis.

The dry urea-agent may be provided as pre-weighed and pre-determined amount encased in capsules for mixing with a pre-determined volume of the diluent. The capsules may be open-able type, for opening and dispensing of the dry urea-agent inside the capsule for mixing and re-hydration with the diluent (of predetermined volume). The capsule may be a blister type materials, for example blister type packaging used for medicine or in pharmaceutical industry, The capsule for the dry urea-agent is preferably made of cellulose, plastic, metallic foil, or other non-reactive agents.

When the dry urea-agent is provided in capsules, the capsules containing the dry urea-agent is, preferably, packed (packaged) with a means to maintained and/or minimize the humidity around the capsules at or below ambient. The humidity maintaining means may be desiccant materials (as bags of desiccants, crystals, etc.) . The capsule may be individually packed in blister containers.

The dry urea-agent may be provided as pre-weighed and pre-determined amount encased in sachets (envelops or bags), for mixing with a pre-determined volume of the diluent.

### PREFERRED EMBODIMENTS OF THE INVENTION

This invention is further explained with the help of the following examples. The following examples consists of seven different compositions of the protein solubilization reagent. Each protein solubilization reagent is prepared in two parts, a dry urea-agent and a diluent for re-hydration (hydration) of the dry urea-agent according to the instruction provided with the protein solubilization reagent.

The dry urea-agent contains urea. The dry urea-agent may also contain other dry chemical agents non-reactive with the urea, such as dry thiourea, detergents, sufobetaines, sulfonates, buffer agents, or dyes. Preferably, the dry urea-agent contains urea, thiourea, detergents, sufobetaines, sulfonates, and buffer agents. Preferably, the detergents, sufobetaines, and sulfonates added into dry urea-agent is selected from ASB-14, ASB-16, ND SB 201, CHAPS, and SB 3 10 The components of the dry urea-agent are mixed together and ground to homogeneity and into fine powder so that the constituent agents do not separate on standing.

For achieving urea concentration 8-9M and thiourea concentration 2M on hydration (rehydration), the dry urea-agent contains urea and thiourea in the following proportion—each 100 gm of urea is mixed with 41-43 gm thiourea. Furthermore, for achieving 2% concentration of additional agents, such as detergents, sufobetaines, and sulfonates—each 100 gm of urea is mixed with 41-43 gm thiourea, 5-6 gm of additional agent(s). For achieving 4% concentration of additional agents, each 100 gm of urea is mixed with 41-43 gm thiourea, 10-12 gm of additional agent(s). For examples, read the composition of Protein Solubilization Reagent, III, IV, VI and VII.

It should be noted and appreciated that the concentration and the molarity of the individual agents mentioned in this invention are meant to be approximate and small variations or modifications are tolerated in this field. For example, each 100 gm of urea may be mixed with 40-44 gm of thiourea and 4-7 gm of additional agents (to achieve 2% final concentration of the additional agent) or 9-13 gm of additional agents (to achieve 4% final concentration of the additional agent)—such variations and modifications will be tolerated in this field.

The diluent is water and may contain, buffering agents, dyes, carrier ampholyte, detergents, glycerols, reducing agents, and alkalation agents. Preferably, the diluent contains water, detergents, and buffering agents. Preferably, the detergent is selected from NP-40, ASB 16, ASB 14, SB 3 10, ND SB 201, CHAPS, and other solubilizing agents that may be developed in future. For achieving a hydrated final concentration (for example 2%) of an agent, the agent is added into the diluent at approximately two fold higher concentration than needed at final concentration after hydration (i.e., ˜4%) with the dry urea-agents. For example, read the composition of Protein Solubilization Reagent-I to VI. The diluent is prepared by mixing all of the needed agents in pure water. The diluent is filtered and store separately.

The instruction for preparing working protein solubilization reagent is provided with the dry urea-agent and/or with the diluent. The instruction specifies the volume of the diluent needed for each gram (or capsule) of the dry urea-agent (or their equivalent) to achieve a desired final concentration of each constituent agents on hydration (re-hydration).

When the dry urea-agent is provided in capsules (or bags, sachets, blisters), the capsule (or bags, sachets, blisters) is, preferably, opened (by pulling apart the two halves of the capsule shell) and the content of the capsule (the day urea-agent) is transferred (emptied) into another container for re-hydration with the diluent.

### EXAMPLES

The following are a few examples of the urea based protein solubilization reagent. Each protein solubilization reagent is prepared in two parts, a dry-urea agent and a diluent for re-hydration of the dry-urea agent. After re-hydration of the dry-urea agent each protein solubilization reagent has a distinct composition.

Protein Solubilization Reagent-I

Protein Solubilization Reagent-I, contains a dry urea-agent and a diluent. The dry urea-agent contains—urea and the diluent contains—NP-40.

The final concentration of the individual agents after mixing (re-hydration) of the two parts of the protein solubilization reagent-I are; 8-9M Urea and 4% NP-40.

Dry-urea agent contains only dry urea powder. The diluent contains 7% solution of NP-40.

Mixing and re-hydration instruction—for each 1 gm dry urea-agent add approximately 1.15 ml diluent.

Protein Solubilization Reagent-I

Protein Solubilization Reagent-II, contains a dry urea-agent and a diluent. The dry urea-agent contains- urea and the diluent contains- CHAPS.

The final concentration of the individual agents after mixing (re-hydration) of the two parts of the protein solubilization reagent-II are; 8-9M Urea and 4% CHAPS.

Dry-urea agent contains only dry urea powder. The diluent contains 7% solution of CHAPS.

Mixing and re-hydration instruction—for each 1 gm dry-urea agent add approximately 1.15 ml diluent.

Protein Solubilization Reagent-III

Protein Solubilization Reagent-III, contains a dry urea-agent and a diluent. The dry urea-agent contains- urea, thiourea, and ASB-16 and the diluent contains—CHAPS.

The final concentration of the individual agents after mixing of the two parts of the protein solubilization reagent-III are; approximately 6-8M Urea, 2M Thiourea, 2% ABS-16, and 2% CHAPS.

Dry-urea agent contains urea, thiourea, and ASB-16, in the following proportion—each 100 gm of urea is mixed with 41-43 gm thiourea and 5-6 gm ABS-16. The diluent contains 4% solution of CHAPS.

Mixing and re-hydration instruction—for each 1 gm dry-urea agent add approximately 1 ml diluent.

For example; in dry urea-agent, 360 gm of urea is mixed with 152 gm of thiourea and 20 gram of ABS-16 and the diluent contains 4% solution of CHAPS made in pure water. After blending the dry urea-agent, 2.5 gm of the dry-urea agent is re-hydrated with 2.5 ml of the diluent, the final volume of the re-hydrated Reagent-III is ˜4.5 ml.

Protein Solubilization Reagent-IV

Protein Solubilization Reagent-IV, contains a dry urea-agent and a diluent. The dry urea-agent contains- urea, thiourea, and SB 3 10 and the diluent contains- CHAPS.

The final concentration of the individual agents after mixing of the two parts of the protein solubilization reagent-IV are; approximately 6-8M Urea, 2M Thiourea, 2% SB 3 10, and 2% CHAPS.

Dry-urea agent contains urea, thiourea, and SB 3 10, in the following proportion—each 100 gm of urea is mixed with 41-43 gm thiourea and 5-6 gm SB 3 10. The diluent contains 4% solution of CHAPS.

Mixing and re-hydration instruction—for each 1 gm dry-urea agent add approximately 1 ml diluent.

Protein Solubilization Reagent-V

Protein Solubilization Reagent-V, contains a dry urea-agent and a diluent. The dry urea-agent contains—urea and thiourea and the diluent contains—CHAPS.

The final concentration of the individual agents after mixing of the two parts of the protein solubilization reagent-V are; approximately 6-8M Urea, 2M Thiourea, and 4% CHAPS

Dry-urea agent contains urea and thiourea, in the following proportion—each 100 gm of urea is mixed with 41-43 gm thiourea. The diluent contains 7% CHAPS.

Mixing and re-hydration instruction—for each 1 gm dry-urea agent add approximately 1.15 ml diluent.

For example; in dry urea-agent, 360 gm of urea is mixed with 152 gm of thiourea and the diluent contains 7% solution of CHAPS made in pure water. After blending the dry urea-agent, 2.5 gm of the dry-urea agent is re-hydrated with 2.87 ml of the diluent, the final volume of the re-hydrated Reagent-V is ˜4.5 ml.

Protein Solubilization Reagent-VI

Protein Solubilization Reagent-VI, contains a dry urea-agent and a diluent. The dry urea-agent contains—urea, thiourea, ND SB 201 and ASB-16 and the diluent contains—CHAPS. The final concentration of the individual agents after mixing of the two parts of the protein solubilization reagent-IV are; approximately 6-8M Urea, 2M Thiourea, 2% ND SB 201, 2% ASB-16, and 2% CHAPS.

Dry-urea agent contains urea, thiourea, ND SB 201 and ASB-16, in the following proportion—each 100 gm of urea is mixed with 41-43 gm thiourea and 5-6 gm each of ASB-16 and ND SB 201. The diluent contains 4% solution of CHAPS.

Mixing and re-hydration instruction—for each 1 gm dry-urea agent add approximately 1 ml diluent.

For example; in dry urea-agent, 360 gm of urea is mixed with 152 gm of thiourea and 20 gram each of ABS-16 and ND SB 201. The diluent contains 4% solution of CHAPS made in pure water. After blending the dry urea-agent, 2.5 gm of the dry-urea agent is rehydrated with 2.5 ml of the diluent, the final volume of the re-hydrated Reagent-VI is ˜4.5 ml.

It should be noted and appreciated that in the above examples (I to VI), CHAPS has been added in the diluent. In an alternative embodiment, CHAPS may be added in dry urea-agent. In such embodiment, the diluent contains pure water, other agents may be added in water such as buffering agents and dyes. Furthermore, the concentration and the molarity of the individual agents mentioned in this invention are meant to be approximate and small variations are tolerated in this field.

Protein Solubilization Reagent-VII

Protein Solubilization Reagent-VI, contains a dry urea-agent and a diluent. The dry urea-agent contains—urea, thiourea, and ND SB 201 and the diluent contains—CHAPS.

The final concentration of the individual agents after mixing of the two parts of the protein solubilization reagent-IV are; approximately 6-8M Urea, 2M Thiourea, 2% ND SB 201, and 2% CHAPS.

Dry-urea agent contains urea, thiourea, and ND SB 201, in the following proportion—each 100 gm of urea is mixed with 41-43 gm thiourea and 5-6 gm ND SB 201. The diluent contains 4% solution of CHAPS.

Mixing and re-hydration instruction—for each 1 gm dry-urea agent add approximately 1 ml diluent.

Protein Solubilization Reagent-VIII

Protein Solubilization Reagent-VII, contains a dry urea-agent and a diluent. Dry urea-agent contains—Urea, SDS, and Diluent contains—water, EDTA, Starch, Tris, Glycerol, and BPB.

The final concentration of the individual agents after mixing of the two parts of the protein solubilization reagent are; approximately 6-8M Urea, 2% SDS, EDTA (1 mM), Starch (0.1%), 0.2 M Tris pH 8.7-8.8, 30% Glycerol, and 0.001% BPB.

Dry urea-agent contains urea and SDS, in the following proportion—each 100 gm of urea is mixed with 5-6 gm SDS. The diluent contains water, EDTA, starch, tris, pH 8.5-8.8, glycerol, and BPB.

Mixing and re-hydration instruction—for each 1 gm dry-urea agent add approximately 1.4-1.8 ml diluent.

For example; in dry urea-agent, 360 gm of urea is mixed with 20 gram of SDS. The diluent contains, 0.2M Tris, pH 8.5-8.8, 1 mM EDTA, 0.1% potato starch, 30% glycerol, and 0.001% bromophenol blue. After blending the dry urea-agent, 2.5 gm of the dry-urea agent is rehydrated with 3.5 ml of the diluent, the final volume of the re-hydrated Reagent-VIII is ˜5.2 ml.

The dry urea-agent part of the protein solubilization reagents (I-VIII) are either packaged in bottles or jars (containing 50-100 gm) or in capsules (containing 0.25 gm to 1 gm each capsule). When the dry urea-agent is packaged in capsules (or bags, sachet, or blisters) for re-hydration the capsules (or bags, sachet, or blisters) are, preferably, opened and the content of the capsules are transfer to another tube for re-hydration with the corresponding diluent. The capsule may also be directly suspended in the diluent, provided that the capsules are made of non-protein materials that dissolve in diluents, such as cellulose and other similar materials.

The full chemical names of the chemical agents used in protein solubilization reagent are CHAPS: [3-(3-cholamidopropyl)dimethylammonio]-1 propane sufonate

NP-40: Nonidet NP-40

ASB 16: aminosulfobetaine-16

ND SB 201: [3-(1-pyridinol)-1-propane sulfonate

SB 3 10: N-decyl-N,N-dimethyl-3-ammonio-1-propane sulfonate (also know as caprylyl sufobetaine)

1. A protein solubilization reagent, comprising:
a dry urea-agent in a container;
a diluent for re-hydration of the dry urea-agent; and
an instruction for combining a pre-determined volume of the diluent with a predetermined amount of the dry urea-agent.

2. The protein solubilization reagent according to claim 1, wherein the dry urea-agent comprises of urea and one or more agents selected from a group consisting of dry thiourea, a detergent, a sufobetaine, a sulfonate, a buffering agent, and a dye.

3. The protein solubilization reagent according to claim 1, wherein the diluent comprises of water and one or more agents selected from a group consisting of a dye, a glycerol, a detergent, a carrier ampholyte, and a buffering agent.

4. The protein solubilization reagent according to claim 1, wherein approximately 1-2 ml of the diluent is mixed with approximately per one gram dry weight of the dry urea-agent to achieve urea concentration approximately 6-9M in the re-hydrated buffer.

5. The protein solubilization reagent according to claim 1, wherein approximately 1-2 ml of the diluent is mixed with approximately per one gram dry weight of the dry urea-agent to achieve urea concentration approximately 6-8M and/or thiourea concentration approximately 2M in the re-hydrated buffer.

6. The protein solubilization reagent according to claim 1, wherein the container for the dry urea-agent is a capsule.

7. The protein solubilization reagent according to claim 6, wherein the capsule is an open-able capsule.

8. The protein solubilization reagent according to claim 1, wherein the container for the dry urea-agent is a sachet.

9. The protein solubilization reagent according to claim 1, wherein the dry urea-agent each 100 gram urea is mixed with 41-43 gram of thiourea.

10. The protein solubilization reagent according to claim 1, wherein the dry urea-agent each 100 gram urea is mixed with 41-43 gram of thiourea and 5-6 gm of an agent selected from a group consisting of a detergent, a sufobetaine, and a sulfonate.

11. The protein solubilization reagent according to claim 1, wherein the dry urea-agent each 100 gram urea is mixed with 41-43 gram of thiourea and 10-12 gm of an agent selected from a group consisting of a detergent, a sufobetaine, and a sulfonate.

12. The protein solubilization reagent according to claim 1, wherein the dry urea-agent is provided in tablet form.

13. A protein solubilization reagent, for treatment of protein, comprising:
a dry urea-agent in a capsule, wherein the urea-agent consists of urea and one or more agents selected from a group consisting of dry thiourea, a detergent, a sufobetaine, a sulfonate, a buffering agent, and a dye.

14. A protein solubilization reagent, for treatment of protein, comprising:
a dry urea-agent in a container, wherein the urea-agent comprises of urea and one or more agents selected from a group consisting of dry thiourea, a detergent, a sufobetaine, a sulfonate, a buffering agent, and a dye; and
an instruction for re-hydration of the urea-agent to achieve urea concentration 6-9 molar and/or thiourea concentration approximately 2 molar.

15. The protein solubilization reagent according to claim 14, wherein the container for the dry urea-agent is a capsule.

16. The protein solubilization reagent according to claim 15, wherein the capsule is an open-able capsule.

17. The protein solubilization reagent according to claim 15, wherein the capsule is packaged with a means to maintain humidity at or below ambient.

18. The protein solubilization reagent according to claim 14, wherein the dry urea-agent each 100 gram urea is mixed with 41-43 gram of thiourea.

19. The protein solubilization reagent according to claim 1, wherein the dry urea-agent each 100 gram urea is mixed with 41-43 gram of thiourea and 5-6 gm of an agent selected from a group consisting of a detergent, a sufobetaine, and a sulfonate.

20. The protein solubilization reagent according to claim 1, wherein the dry urea-agent each 100 gram urea is mixed with 41-43 gram of thiourea and 10-12 gm of an agent selected from a group consisting of a detergent, a sufobetaine, and a sulfonate.
